(19) United States
(12) Patent Application Publication
(10) Pub. No.: US 2007/0255256 A1
(43) Pub. Date: Nov. 1, 2007

(54) Irrigation Catheter

(75) Inventors: Frank J. Fischer JR., Bloomington, IN (US); Thomas L. Foster, Poland, IN (US); Tracy E. Willis, Bloomington, IN (US)

Correspondence Address: BRINKS HOFER GILSON & LIONE/CHICAGO/COOK, PO BOX 10395, CHICAGO, IL, 60610, US

(73) Assignee: Vance Products Incorporated, Spencer, IN (US)

(21) Appl. No.: 11/547,537

(22) PCT Filed: Mar. 24, 2005
(86) PCT No.: PCT/US05/09849

§ 371 Date: Jun. 19, 2007

Related U.S. Application Data
(60) Provisional application No. 60/557,547, filed on Mar. 30, 2004.

(51) Int. Cl. A61M 25/00

(52) U.S. Cl. 604/528

(57) ABSTRACT

An irrigation catheter having at least two lumens for supplying irrigation fluid in minimally-invasive surgical procedures is revealed. The catheter works alongside but independently of an endoscope or other diagnostic or therapeutic instrument. The catheter may direct flow of the irrigation fluid in the same direction as the body's natural flow, thus enhancing visualization by the endoscope or other instrument.

This application claims the benefit of the filing date under 35 U.S.C. § 119(e) of Provisional U.S. patent application Ser. No. 60/557,547, filed on Mar. 30, 2004, which is hereby incorporated by reference in its entirety.

### FIELD OF THE INVENTION

The field of the invention is medical devices, especially medical devices used in minimally-invasive procedures. The particular field of the invention is that of catheters and devices used to provide irrigation and cleansing of optical instruments used for diagnostic or therapeutic purposes.

### BACKGROUND

Minimally invasive procedures have become widely accepted in medicine, because some of these procedures enable surgeons to accomplish in an office and outpatient setting procedures that formerly required overnight stays in a hospital. Other procedures that still require a hospital stay are now accomplished with less trauma to the patient than that caused by major surgical procedures. The procedures are generally accomplished by using natural openings or orifices in the body. If a natural orifice is not suitable, an orifice is created in a manner that minimizes the trauma to the patient.

A consequence of these procedures is that the surgeon is dependent on remote manipulation in the operating field and remote visualization of the operating field. Thus, endoscopes and endoscopic instruments, typically used in a working channel of an endoscope, are widely used in minimally-invasive procedures. The endoscope typically includes an optical system that enables the surgeon to see the operating field. The optical system includes a source of light and lens or camera to capture the reflected light and return the image to the surgeon.

One problem that has arisen with optical systems is the need for irrigation within the operating field. A supply of irrigation fluid, such as saline solution, is used to clear the lens or camera input of blood and other matter so that the surgeon may continue to visualize the operating field and complete the procedure. The easiest way to provide an irrigation system is to use one of the channels of the endoscope, i.e., a tube or catheter for irrigation is placed through one of the working channels of the endoscope. This enables simultaneous movement of both the endoscope and the irrigation source, thus ensuring that the irrigation source will be kept close to the lens or camera and continues to keep it clean.

Using the working channel, however, works several disadvantages. The irrigation source is limited to the size of the working channel, and consequently may be limited in its flow rate. The endoscope or other visualization instrument is similarly limited, in that the instrument must reserve a channel for the irrigation system, in addition to any other channels needed for therapeutic or diagnostic purposes. These may include, for instance, a working channel for manipulating a bioptome to take a biopsy sample, or for manipulating a grasper for removing undesirable objects, such as kidney stones or fragments of kidney stones. Another disadvantage is that the flow of irrigation fluid in these arrangements is counter to the desired flow: in a ureteroscope, for example, the flow is toward the ureter and the kidney, rather than the desired flow, in the body's natural direction, away from the kidney and ureter, toward the bladder, sweeping past the lens and clearing the lens in a more natural direction.

What is needed is a system that will provide irrigation fluid without being limited by the size of a working channel of the endoscope, and which will not prevent endoscopes from continuing to reduce their size or diameter, thus making minimally invasive procedures even easier. What is also needed is an irrigation system in which the flow of fluid is in the same direction as the body's natural flow, and which flow will continue to clear the lens and optical system.

### BRIEF SUMMARY

One aspect of the invention is an irrigation catheter for use with a diagnostic or therapeutic device, the irrigation catheter including a connector The irrigation catheter also includes an elongated tube connected to the connector for conveying irrigation fluid, the tube including a distal portion and a proximal portion stiffer than the distal portion, wherein the tube further comprises at least two lumens each lumen having an orifice.

Another aspect of the invention is a double lumen irrigation catheter. The double lumen irrigation catheter includes an elongated portion including a first tube and a second tube. The double lumen catheter also includes at least one connector at a proximal end of the elongated portion, and also includes a distal portion further including at least one orifice, wherein the irrigation fluid flows in a first direction from a source of irrigation fluid and is directed in a second direction by the distal portion and the at least one orifice.

Another aspect of the invention is a ureteroscopy irrigation catheter for use outside a ureteroscope, the irrigation catheter including a connector and an elongated portion comprising at least two lumens connected to the connector. The ureteroscopy irrigation catheter also includes a distal end, the distal end designed to at least partially block a body orifice and directing a flow of irrigation fluid in a direction different from the direction of inflow of the irrigation fluid in the elongated portion.

Another aspect of the invention is a method of manufacturing an irrigation catheter, the method including molding the catheter, the catheter including at least two lumens, a distal end and an elongated portion; and connecting a connector to the tubular portion.

Another aspect of the invention is a method of using an irrigation catheter. The method includes providing an irrigation catheter having at least two lumens, at least one lumen connected to a connector for conveying irrigation fluid, the catheter including a proximal portion and a distal portion, wherein the catheter conveys irrigation fluid in a first direction and the distal portion further includes at least one orifice for directing the irrigation fluid in a second direction. The method also includes placing the irrigation catheter through an endoscopic instrument or an access sheath, orienting the irrigation catheter to control placement of a flow of irrigation fluid, and operating the irrigation catheter.

These and other aspects of the invention are described below. The following drawings form part of the present application and are included to further demonstrate certain aspects of the present invention. The invention may be better understood by reference to one or more of these drawings in combination with the detailed description of specific embodiments presented herein.

### BRIEF DESCRIPTION OF THE DRAWINGS

FIGS. 1 and 2 are perspective views of the prior art;

FIG. 3 is a broken, partial cross-sectional view of a first embodiment;

FIG. 4 is a broken, partial cross-sectional view of a second embodiment;

FIG. 5 is a broken, partial cross-sectional view of a third embodiment;

FIG. 6 is a broken, partial cross-sectional view of a fourth embodiment;

FIG. 7 is a perspective view of the fourth embodiment in use with an endoscope.

FIG. 8 is a broken, partial cross-sectional view of a fifth embodiment;

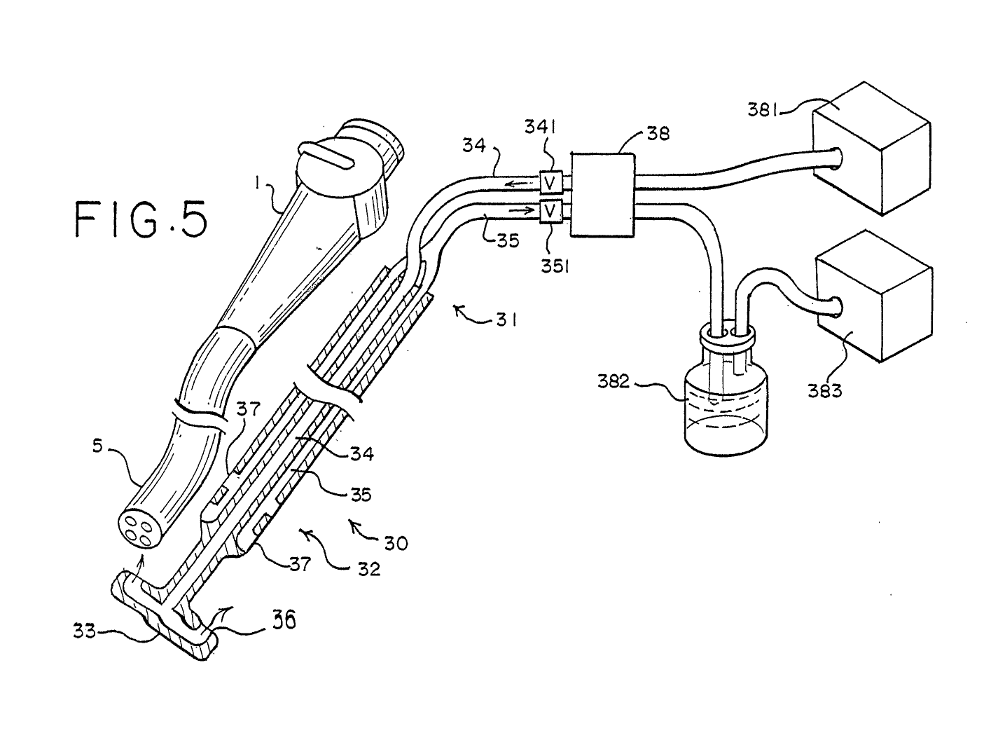

FIG. 5a is a closer perspective view of a multi-lumen embodiment; and

FIG. 9 is a broken, partial cross-sectional view of a sixth embodiment.

### DETAILED DESCRIPTION OF THE DRAWINGS AND THE PRESENTLY PREFERRED EMBODIMENTS

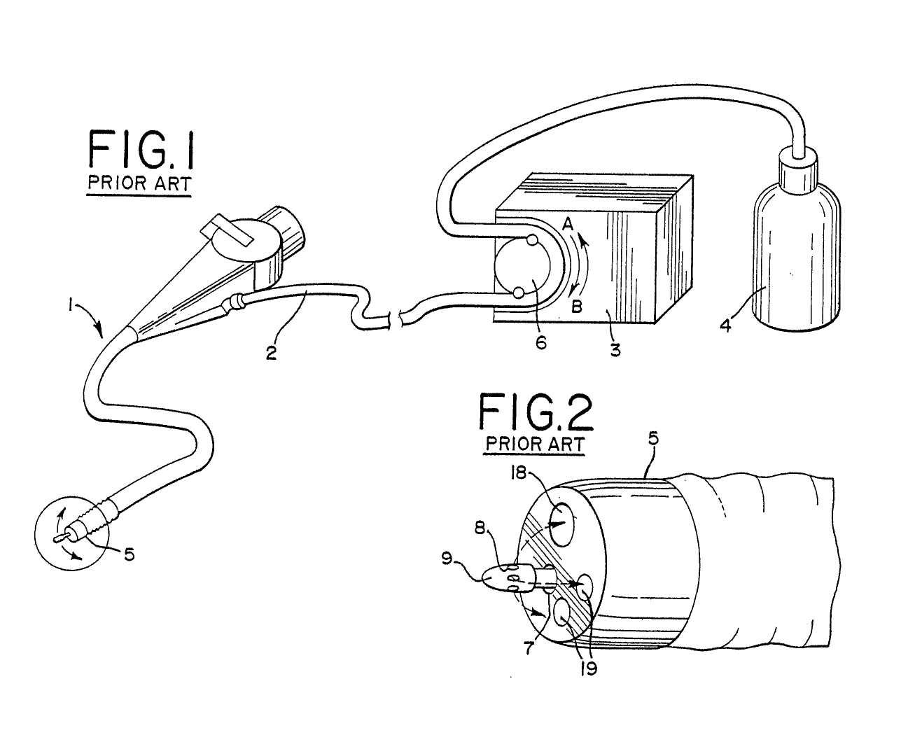

Prior art systems for providing irrigation for an endoscope are depicted in FIGS. 1 and 2. In FIG. 1, an endoscopic instrument 1 is equipped with an irrigation channel 2 for irrigating the distal end 5 of the instrument. The irrigation fluid may be supplied by pump 3 from a bottle or container 4 of irrigation fluid. The pump may include a peristaltic mechanism 6 or other mechanism suitable for delivering the fluid. Gravity alone may be suitable, if the source of irrigation fluid is, for instance, a saline irrigation bag held high enough to supply sufficient head for the desired flow.

FIG. 2 depicts the distal end 5 of the endoscope Irrigation channel 2 is inserted in a working channel 7 of the endoscope. The channel terminates in a nozzle 9 and orifices 8 which supply irrigation fluid for clearing light sources 19 and lens 18 of the endoscope. As seen in FIGS. 1-2, the nozzle diameter is limited to the diameter of working channel 7. The irrigation channel 2 and nozzle 9 is also limited to the close proximity of distal end 5. This system also provides very little control for the direction of the fluid, and does not provide for collection of the irrigation fluid for removal from the patient.

Embodiments of the present invention may include single-lumera or double lumen catheters, and the catheters may be equipped for communication with a source of irrigation fluid, or with both a source of fluid and a source of egress, such as a vacuum. It is preferred that the proximal end of the catheter be equipped with a fitting, to attach the source of fluid, or the sources of fluid and vacuum. It is also preferred that the distal end of the catheter, whether single or double lumen, have a diameter or configuration greater than the diameter of the elongated tube that includes the greatest portion of the catheter.

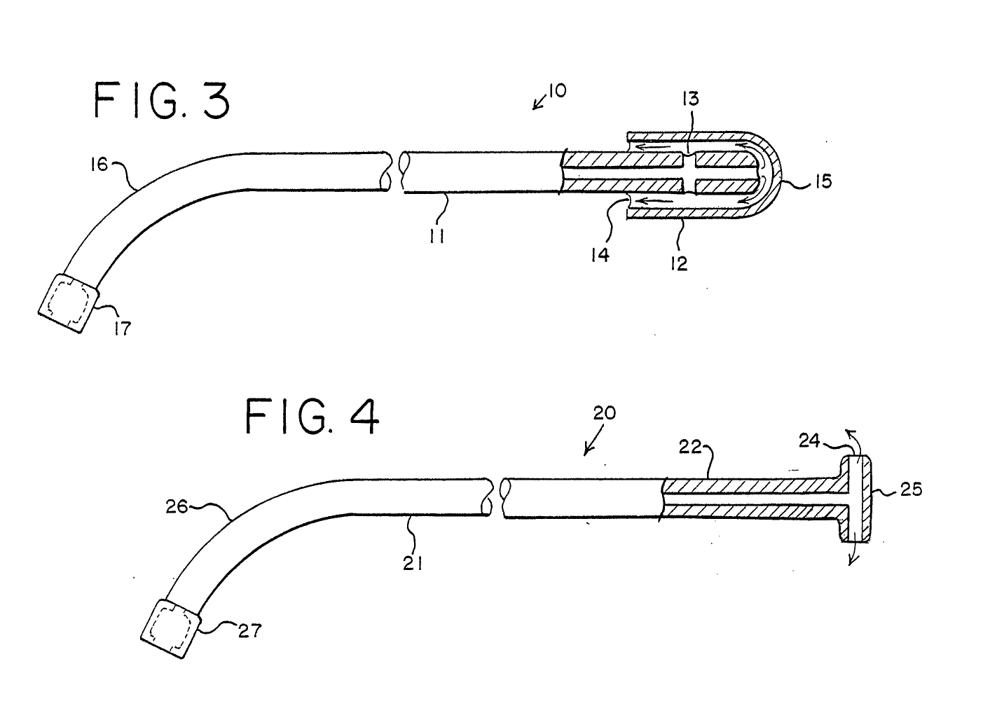

A first embodiment of an irrigation catheter is depicted in FIG. 3. Catheter 10 includes a proximal portion 16 with connector 17, which may be a Luer lock connector or other connector suitable for communication with a source of irrigation fluid. Catheter 10 includes an elongated tube 11 for most of its length, and terminates in distal portion 12. Distal portion 12 includes orifices 13 from elongated tube 11 to distal tip 15. Distal tip 15 also include orifices 14 directing a flow of irrigation fluid counter or opposite to the direction of flow in elongated tube 11.

The catheter may be described as a hollow tube with a boot added at the distal end to re-direct the flow of fluid. It will be appreciated that catheter 10 may be used for irrigation of a ureteroscope. In this case, distal portion 12 will be nearer to a kidney or a ureter of a patient, and the direction of flow of the irrigation fluid when it leaves distal tip 15 will follow the natural flow of urine outward, in the direction from the kidney through the ureter and to the bladder. It will be appreciated that different sizes and embodiments of catheter 10 may be used with endoscopic instruments other than ureteroscopes. The distal tip or boot is preferably somewhat stiffer than the elongated tube that includes the greatest portion of the irrigation catheter. The distal tip may be made stiffer by using a material with a higher modulus of elasticity or by making the distal tip a little thicker than the rest of the catheter.

In one procedure using the catheter, an endoscope of outer diameter 9.5 Fr is inserted into a patient though a Flexor® ureteral access sheath, available from Cook Urological Incorporated, Spencer, Ind. Other useful sheath diameters may include 12 and 14 Fr. The irrigation catheter is then inserted through the access sheath and positioned so that the distal end of the catheter abuts the opening of the ureter into the bladder. The surgeon may use the endoscope to verify placement of the endoscope and the catheter, and may then begin the procedure.

The configuration of the catheter at the distal portion is somewhat squat, designed for distal tip 15 to rest in the area of the ureter and to at least partially block flow of the irrigation fluid from entering the ureter. Some sizes or embodiments may be designed so that distal tip 15 may be placed further into the ureter. Larger sizes may be designed for placing the distal tip in the area where the ureter meets the bladder. In both of these applications, the catheter will direct flow of irrigation fluid into the desired region, and then turn the flow about 180°.

In this way, the irrigation fluid will be directed to flow in a manner consistent with body fluids, from kidney to ureter and bladder. Thus, any undesirable materials, such as kidney stones or fragments, will tend to be washed out of the body, rather than further into the ureter and kidney. The irrigation fluid used will have to be removed sooner or later during the procedure. With this invention, the fluid can irrigate the optical system, allow the physician to view the operating field, and tend to help remove items from the body, rather than exacerbating the problem of removal of fluid and particles. The distal end of the irrigation catheter is preferably designed so that it will fit into the ureter, or other body passage, and at least partially block the flow of irrigation fluid away from the area of the catheter and endoscope for which the irrigation is desired.

Another embodiment of the invention is depicted in FIG. 4. Irrigation catheter 20 includes an elongated tube 21, a proximal portion 26, and a fitting 27 at the proximal end of the catheter so that a source of irrigation fluid may be attached. Catheter 20 also includes a distal portion 22 and a distal end 25. Distal end 25 has a diameter somewhat larger than the elongated tube portion, and also has orifices 24 to direct the irrigation fluid to flow counter to the direction of flow in elongated tubular portion 21. The number and size of the orifices may be varied as desired, so long as the desired flow characteristics are achieved.

The components for the irrigation catheter may be made from plastic. The fitting and the catheter may be PVC, or may be made from any other medically-acceptable flexible material, such as silicone, C-flex (thermoplastic elastomeric material), polypropylene, polyethylene, polyurethane, nylon, or fluorocarbon materials, such as polytetrafluoroethylene. Tubing available from United States Plastic Corp., Lima, Ohio, in several materials and several grades and sold under the brand name of “Tygon® tubing” may also be used. The fitting at the proximal end may be made from any of a variety of materials, including PVC, ABS, polycarbonate and acrylic. ABS is preferred, but other plastics may be used, such as polycarbonate or acrylic.

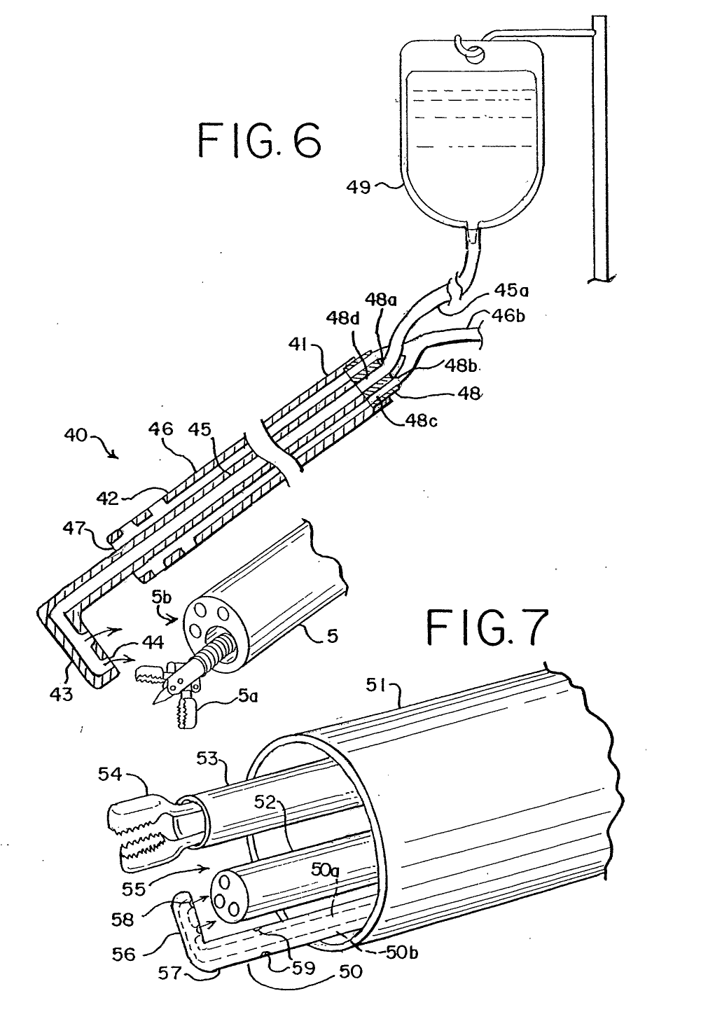

Other embodiments of the irrigation catheter may use a double-lumen elongated portion, as depicted in FIGS. 5-6. FIG. 5 depicts an endoscopic instrument 1 with distal end 5 used with a double-lumen irrigation catheter 30 having inner lumen 34 and outer lumen 35. The catheter includes a proximal portion 31 and a fitting 38, along with two check valves 341, 351. The check valves assure that the fittings cannot be hooked up backwards. The fitting 38 may include two fittings, to connect catheter 30 to a source of irrigation fluid 381 and a collection facility 382 and a source of egress, such as a vacuum 383.

Inner lumen 34 terminates in a distal end 33, in which the flow of irrigation fluid is reversed when the fluid flows from backwards-facing orifices 36. The fluid flows counter to the direction of the fluid in inner lumen 34. Orifices 36 are preferably also directed to cleansing and clearing the lens or other desired portion of an optical system at distal end 5 of endoscopic instrument 1. The irrigation fluid thus flows in a direction to leave the body of the patient. The fluid is collected and drained by the catheter through orifices 37 leading to outer lumen 35. Lumen 35 will be an area of lower pressure even if vacuum is not supplied through fitting 38 because the areas adjoining outer lumen 35 and orifices 37 are areas of higher pressure because of the inflow of irrigation fluid.

One-way or check valves 341, 351 are used to insure that the irrigation fluid flows only one way, i.e., into the catheter through inlet check valve 341 and out through outlet check valve 351 to a collection point or to a source of vacuum. The fluid then flows into and out of the endoscope or other instrument for which irrigation is desired. The valves may be any suitable valves, but those available from Qosina Corp., Edgewood, N.Y. are preferred. In one preferred embodiment, the external portions of the check valves are manufactured in different colors, such as a blue external portion for the inlet valve and a white external portion for the outlet valve. This color coding provides a quick check both for manufacturing personnel and for operating-room personnel that the irrigation system for the endoscope or other diagnostic or therapeutic system has been properly assembled.

The irrigation system need not be symmetrical, but may instead have a distal portion that provides irrigation on one side. This may allow for the more precise placement of the flow of irrigation fluid. FIG. 6 depicts a double-lumen irrigation catheter 40 used in conjunction with endoscopic instrument distal end 5 and end-effector forceps 5a and optical system 5b. Catheter 40 has a distal end 43 that is asymmetric with respect to the inner lumen 45 and outer lumen 46 of the catheter. Catheter 40 includes a proximal portion 41 with connector 48 to a source of irrigation fluid 49. Connector 48 also connects to a source of egress or vacuum. Connector 48 may include passages 48a, 48b, for tubes 48c, 48d in connector 48. Tube 48c may lead to another tube 45a for connecting to a source of irrigation fluid 49. Tube 48d may lead to another tube 46b for connection to a collection point for the irrigation fluid and a source of vacuum or drain for the fluid.

Distal portion 42 includes distal end 43 with orifices 44 to direct flow in a direction counter to the inflow of irrigation fluid in the inner lumen 45. Outer lumen 46 includes orifices 47 that may be near the distal end 43 or may be placed on the sides of outer lumen 46 in order to collect the irrigation fluid for removal from the body of the patient. Distal end 43 is preferably a little stiffer or less resilient than the rest of the catheter, so that the distal end resists deformation under the gentle pressure of the irrigation fluid. Even though this pressure is low, probably not more than several inches of water, it is preferable that the irrigation catheter and its distal end are able to maintain their spatial relationship with the optical system that is being cleaned by the irrigation fluid. As discussed above, the catheter may be used with several different kinds of laparoscopic and endoscopic instruments. The surgeon using the instrument will have to make fewer adjustments if the irrigation catheter is steady while irrigating the endoscopic optical system.

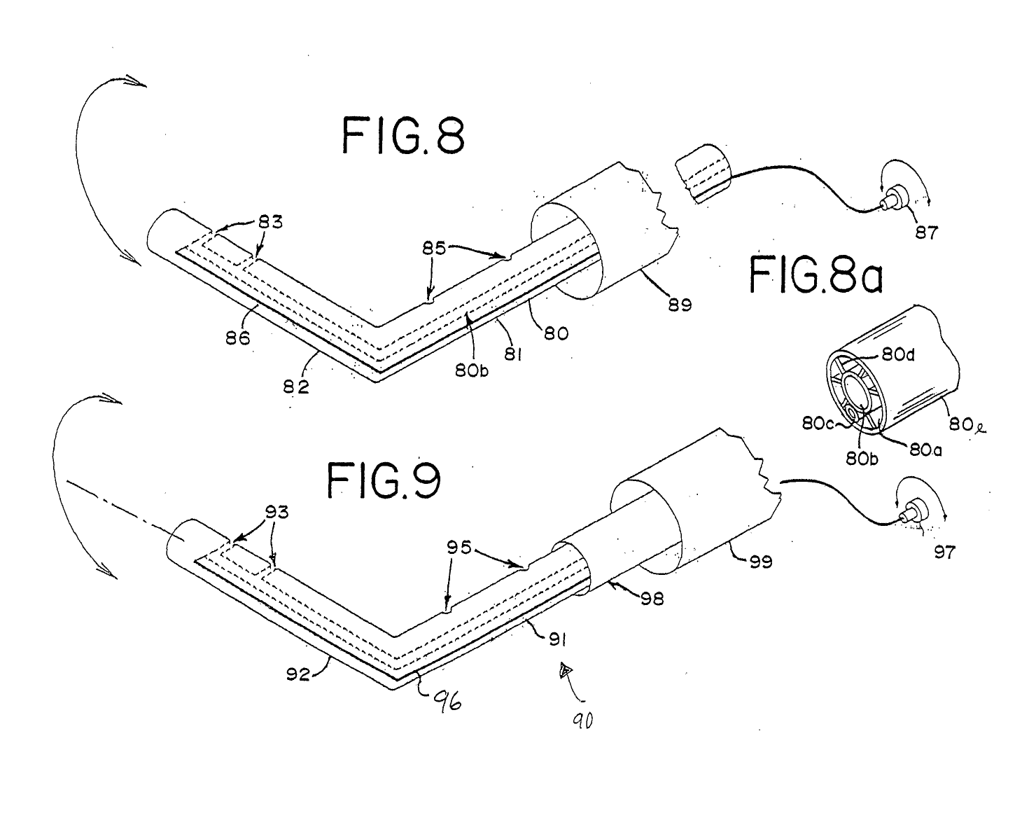

Irrigation catheters of the present invention may be used with instruments other than endoscopes and may also be used with an access sheath, as depicted in FIG. 7. A ureteral access sheath may be introduced into a patient in the usual manner, first by introducing at least one wire guide, followed by the ureteral access sheath 51. Once sheath 51 is in place, trauma to the urethra, bladder, and ureter are minimized. The sheath is then used to insert an endoscope 52, the endoscope preferably having an optical system 55. The surgeon may also use an additional therapeutic or diagnostic instrument, such as a grasper forceps 54.

Optical system 55 may include one or more light sources and a lens or camera for collecting the reflected light and returning an image to the surgeon operating the endoscope. An irrigation catheter 50 is also placed through access sheath 51. Irrigation catheter 50 preferably has two lumens, 50a for inflow of irrigation fluid and an outer lumen 50b for collection and removal of the fluid. Catheter 50 also has a distal portion 57 and an asymmetrical distal end 56 with one or more orifices 58 that direct irrigation fluid in a direction that generally follows the natural flow of body fluids, i.e., towards outside the body. In addition, distal portion 57 has one or more orifices 59 that allow for removal of irrigation fluid through catheter 50. Catheter 50 preferably directs the flow of irrigation fluid in a direction opposite to the direction of influx of fluid, i.e. the irrigation fluid is preferably reversed in direction before it leaves the irrigation catheter. Using an endoscope and the optical system, a surgeon may place the irrigation catheter and orient the catheter to control placement of the irrigation fluid flow. The flow impinges on optical system 55 and eventually flows to outflow orifices 59 for collection and removal from the patient.

It may also be advantageous to use a catheter with more than two lumens. In the embodiments shown in FIGS. 8, 8a, and 9, a third lumen is provided to accommodate a control wire for controlling a direction of the flow of irrigation, or for increasing the rigidity of the catheter. In FIGS. 8 and 8a, a three-lumen catheter 80 may gain access to a patient through an access sheath 89, such as a ureteral access sheath. The surgeon may instead use the working channel of an endoscope for access.

As shown in FIG. 8a, catheter 80 has three lumens, including an outer lumen 80a, an inner lumen 80b, and a third lumen 80c. The third lumen may be placed concentric with the other two lumens, or the third lumen may be placed between the first two, as shown. The three lumens are defined by tubing, such as plastic tubing, on their outer diameters. Webbing 80d, as shown, may be used to increase the rigidity or modulus of catheter 80. If the webbing extends throughout the length of the catheter, the catheter may have more than three lumens. Lumen 80c may instead be used to pass a stylet, a thin wire or rod for maintaining rigidity, through the catheter for straightening. The stylet is preferably straight and is preferably made of rigid material, with a higher modulus than the distal portion of the catheter. The stylet diameter is preferably, but not necessarily, 0.018 to 0.038″ inches. The stylet should have a rounded tip to prevent damage to the catheter. The length of the stylet may be adjusted in accordance with the length of the catheter. In some embodiments, the walls of lumen 80c may be somewhat thicker to maintain their integrity when use of a stylet is anticipated.

The purpose of this particular embodiment of a three-lumen catheter is for a preformed control wire 86 to be placed within the third lumen 80c, so that a user can use the control wire to adjust or to aim a flow of irrigation fluid from the catheter to a point or an area as desired, with respect to the catheter or to the patient. At the distal end, catheter 80 includes a proximal portion 81 and a distal portion 82. Inner lumen 80b has orifices 83 for directing a flow of fluid from the catheter to a visual system of the endoscope (not shown) or other desired area. Outer lumen 80a has orifices 85 for gathering fluid from the operating field and routing the fluid outside the patient. Wire 86, preferably with a 90° pre-formed bend as depicted, is connected to a control instrument 87 for rotating the wire, and thus catheter 80, to direct a flow of fluid into or out of the patient. The control wire need not be used only for rotating or directing the catheter, but instead may simply be used to stiffen the catheter. The third lumen may also be used with a previously-placed guide wire to direct the catheter to the desired area within a patient.

The walls of the catheter and of the lumens, such as tubing, may be different in proximal portion 81 from the walls of the catheter and the lumens in distal portion 82. When control wire 86 is rotated using controller 87, proximal portion 81 will rotate, as the wire remains straight and also rotates, while distal portion 82 will rotate and move in the plane (see arrow A) in response to the user's manipulations. Therefore, distal portion 82 preferably has a lower modulus or rigidity than proximal portion 81. Distal portion 82 will bend and flex and move more readily when the wire is rotated, while proximal portion 81 is stiffer and will remain relatively rigid, enabling a user to better transmit torque through the wire to catheter 80. Sheath 89 preferably has a higher flexural modulus than sheath 80 including wire 86 and all the walls of the sheath, so that catheter 80 will conform at least roughly to the shape of sheath 89 when catheter 80 is within sheath 89.

When wire 86 is inserted into lumen 80c, the wire, preferably with a pre-formed bend, will be sufficient to form the angle as shown, bending distal portion 82 in the desired direction. Thus, the flexural modulus, or resistance to bending, of wire 86 is greater at least than distal portion 82 of the catheter, so that distal portion 82 will assume a molded configuration when distal portion 82 has pushed through sheath 89 and is free to assume its “natural” uncompressed or unstressed state. It is also preferable for proximal section 81 of the catheter to be stiffer, having a higher flexural modulus, than at least the portion of wire 86 that extends to the distal portion 82 of the catheter. Thus, wire 86 would be stiffer than distal portion 82 but less stiff, or more compliant, as compared to proximal portion 81 of the catheter.

Another embodiment of a three-lumen catheter is depicted in FIG. 9. In this embodiment, three-lumen catheter 90 is used within a working channel 99 of an endoscopic instrument; alternatively, access may be gained through an access sheath. Catheter 90 includes an outer sleeve 98 that is relatively less flexible, and has sufficient rigidity to counteract the bending of both catheter 90 and control wire 96. Thus, catheter 90 will remain straight until sleeve 98 is retracted, allowing distal portion 92 to curve or bend, as shown, while proximal portion 91 remains straight. An inner lumen of catheter 90 includes orifices 93 for distributing irrigation fluid, while an outer lumen includes orifices 95 for gathering fluid and routing the fluid outside the operating field. In the instances where catheter 90 and sleeve 98 are used with wire 96 inside an access sheath, such as access sheath 89 in FIG. 8, the access sheath is preferably relatively stiff, so that its resistance to bending, or flexural modulus, is greater than the combined flexural modulus of catheter 90 and wire 96, and sleeve 98.

In this embodiment, wire 96 in the third lumen is pre-formed so that when sheath 98 retracts, wire 96 will have about a 90° bend in distal portion 92. When a user then manipulates controller 97, wire 96 will twist, and the twisting will cause the distal portion 92 of catheter 90 to rotate in-plane, as shown by arrow A in FIG. 9. Irrigation fluid will flow from orifices 93 to a desired location, under the control of controller 97 and the user. While wires and stylets have been mentioned in relation to the embodiments of FIGS. 8, 8a, and 9, control wires and stylets may be used with many embodiments of the invention. Control wires for straightening and orienting the catheter are not limited to irrigation catheters with three lumens, although a wire is most advantageously used when the wire has its own lumen. The wire should not interfere with other functions of the catheter, and the walls of the lumen should be sufficiently thick that there is no possibility of puncture of the wire through the walls.

The invention has been described in terms of embodiments useful especially in irrigation for endoscopic instruments that require visualization during a medical procedure in the area of the kidney, the ureter and the bladder. The invention contemplates structures including and consisting of the embodiments shown in the figures and described in the text. Other embodiments may be used in other areas of the body, such as a biliary duct, a uterus, or an upper gastrointestinal tract. Rather than using an endoscope, other instruments may be used, such as a hysteroscope, a laparoscope, a panendoscope, an esophagoscope, a gastroscope, or a duodenoscope. As noted above, the catheter is preferably used along with a ureteral access sheath or other access device, so that trauma to body tissues is minimized. Other embodiments may be used in other applications and other areas of the body, such as embodiments useful for removing gallstones from the bile duct or related areas. These embodiments are not limited to human bodies, but may be used in veterinary service as well.

It is the intention of the applicants to protect all variations and modifications within the valid scope of the present invention. Many other variations of the invention may also be used without departing from the principles outlined above. It is intended that the invention be defined by the following claims, including all equivalents. Since the foregoing detailed description has described only a few of the many alternative forms this invention can take, it is intended that only the following claims, including all equivalents, be regarded as a definition of this invention.

Accordingly, it is the intention of the applicant to protect all variations and modifications within the valid scope of the present invention. It is intended that the invention be defined by the following claims, including all equivalents.

What is claimed is:

1. An irrigation catheter for use with a diagnostic or therapeutic endoscopic instrument, the irrigation catheter comprising:
a connector; and
an elongated tube connected to the connector, the tube comprising a distal portion and a proximal portion stiffer than the distal portion, wherein the tube further comprises at least two lumens each having at least one orifice.

2. The catheter of claim 1, further comprising a control wire within one of the lumens.

3. The catheter of claim 1, wherein the distal portion is preformed at an angle to the proximal portion.

4. The catheter of claim 1, wherein the lumens are defined by walls and further comprising at least one web between the walls.

5. The catheter of claim 1, wherein the catheter is a triple lumen catheter.

6. The catheter of claim 7, further comprising a rotational controller connected to the control wire.

7. The catheter of claim 1, wherein the distal portion has a diameter different from the proximal portion.

8. The catheter of claim 1, wherein the distal portion is designed to at least partially block a body orifice.

9. A double lumen irrigation catheter, comprising:
an elongated portion comprising a first tube and a second tube;
at least one connector at a proximal end of the elongated portion;
a distal portion further comprising at least one orifice, wherein the irrigation fluid flows in a first direction from a source of irrigation fluid and is directed in a second direction by the distal portion and the at least one orifice.

10. The catheter of claim 9, further comprising at least one check valve connected to the at least one connector.

11. The catheter of claim 9, wherein the second direction is opposite the first direction.

12. The catheter of claim 9, wherein the first tube and second tube are concentric, the first tube inside the second tube, and the second tube further comprises at least two orifices for collection of the irrigation fluid.

13. The catheter of claim 9, wherein the elongated portion is stiffer than, or has a diameter different from, the distal portion.

14. The catheter of claim 9, wherein the first tube and second tube are concentric, the first tube inside the second tube, and the second tube terminates before the distal end.

15. The catheter of claim 9, further comprising a wire in one of the tubes.

16. A ureteroscopy irrigation catheter for use outside a ureteroscope, the irrigation catheter comprising:
a connector;
an elongated portion comprising at least two lumens, at least one lumen connected to the connector; and
a distal end designed to at least partially block a body orifice and directing a flow of irrigation fluid in a direction different from a direction of inflow of the irrigation fluid in the elongated portion.

17. The catheter of claim 16, further comprising at least one check valve connected to the connector.

18. The catheter of claim 16, wherein the elongated portion is stiffer than, or has a diameter different from, the distal end.

19. The catheter of claim 16, wherein the catheter comprises three lumens.

20. The catheter of claim 16, wherein the catheter is a three-lumen catheter, and further comprising a control wire in one of the lumens.

21. The catheter of claim 20, further comprising a controller connected to the control wire.
